(19) United States
(12) Patent Application Publication
(10) Pub. No.: US 2026/0049999 A1
(43) Pub. Date: Feb. 19, 2026

(54) SYSTEMS AND METHODS FOR ASSESSING OXYGEN BINDING IN NATIVE RED BLOOD CELL SUSPENSIONS

(71) Applicant: The Geneva Foundation, Tacoma, WA (US)

(72) Inventors: Orion FURMANSKI, Baltimore, MD (US); Kristin Hedgepath GILCHRIST, Rockville, MD (US)

(73) Assignee: The Geneva Foundation, Tacoma, WA (US)

(21) Appl. No.: 18/801,998

(22) Filed: Aug. 13, 2024

(51) Int. Cl. G01N 33/72; B01L 3/00

(52) U.S. Cl. CPC G01N 33/721; B01L 3/502715; G01N 33/726; B01L 2300/0654; B01L 2300/0663; B01L 2300/0893

(57) ABSTRACT

The disclosure relates to a system and method for assessing red blood cell oxygen affinity involving spectrophotometric oxygen dissociation assay (SODA).

### GOVERNMENT SUPPORT

This invention was made with government support under Contract No. HU00012020011 awarded by the Department of Defense Health Affairs. The government has certain rights in the invention.

### KEYWORDS

Oxygen binding/affinity, Red blood cell suspensions, Oxygen dissociation assay

### BACKGROUND

Oxygen affinity analysis is a crucial component of assessing fitness of patient blood samples or efficacy of transfusable blood products. Red blood cell (RBC) hemoglobin (Hb) oxygen affinity is proportional to both oxygen partial pressure (pO₂) and physiologic pH, such that low pO₂ and pH in distal tissues promote oxygen dissociation. The gold standard of traditional oxygen affinity testing is a dual wavelength spectrophotometer that measures changes in hemoglobin light absorbance as a function of oxygen partial pressure. Standard oxygen affinity testing of RBCs calculates the pO₂ at which 50% of RBC Hb is bound to oxygen (p50) by measuring Hb light absorption patterns during changes in dissolved oxygen. Although standard oxygen affinity analysis systems produce accurate results, they are expensive and typically low-throughput (˜30 minutes for one sample), and p50 calculations are opaque to the user. Furthermore, the high cost of a standard oxygen affinity system presents a barrier to adoption.

### SUMMARY OF THE DISCLOSURE

The disclosure relates to a transparent, low cost, high-throughput system and method for assessing RBC oxygen affinity involving the spectrophotometric oxygen dissociation assay (SODA).

In a first aspect of the disclosure, an exemplary method may include determining oxygen binding affinity to hemoglobin. The method may comprise measuring light absorbance by RBC samples in a liquid suspension. In some embodiments, the method may further comprise analyzing, via a plate reading spectrophotometer (PRS), light passing through a liquid RBC sample. The method may additionally comprise sensing, via at least one probe, oxygen level of the RBC sample. In certain embodiments, the at least one probe may be disposed in a well plate configured to receive the at least one probe. The method may additionally comprise determining oxygen binding affinity for hemoglobin of the RBC sample based on the oxygen level and absorbance data measured by the PRS.

In a second aspect of the disclosure, an exemplary system may determine oxygen binding affinity for hemoglobin. The system may comprise a PRS configured to analyze light passing through a liquid RBC sample. The system may additionally comprise at least one probe. The at least one probe may be configured to measure oxygen level of the RBC sample and may be disposed in a well plate configured to receive the at least one probe. The system may additionally comprise a processor configured to determine oxygen binding affinity for hemoglobin of the RBC sample based on the oxygen level and absorbance data measured by the PRS.

In certain embodiments of the above-disclosed exemplary method and system, the well plate may include wells arranged in columns and rows. In some embodiments, one of the columns and one of the rows of the well plate may have a number of wells smaller than a number of wells in other columns and rows, respectively. In some embodiments, at least two wells of the well plate may define a respective passage that receives at least one probe. In other embodiments, at least one well of the well plate may define an angled port that receives the at least one probe. In some embodiments, the well plate may be fabricated from a 3D printed polycarbonate filament. In certain embodiments, the well plate may be attached to a transparent base plate using a silicone adhesive. In some embodiments, the at least one probe may be configured to measure temperature of the RBC sample. In some embodiments, the absorbance data may be read from a single timepoint in a single well in the well plate and a smooth spectrum of the data may be generated using a low pass filter. In some embodiments, the oxygen binding affinity may be determined by smoothing a log ratio of the absorbance data using a low pass filter, employing a max-min scale log ratio to represent a percent saturation of oxygen and spline interpolation to increase data resolution, and determining an oxygen concentration that corresponds to 50% oxygen saturation.

### BRIEF DESCRIPTION OF THE DRAWINGS

FIG. 1 depicts a schematic of an exemplary system in accordance with the disclosure.

FIGS. 2A-B depict well plates modified for use in SODA analysis.

FIGS. 3A-B depict well plates modified by drilling jigs for use in SODA analysis.

FIG. 4 is a graph depicting absorbance spectra in fully oxygenated and deoxygenated states during introduction of nitrogen flow.

FIG. 5A is a graph depicting Soret band shifts in individual sample wells measured during oxygen depletion. FIG. 5B is a graph depicting Q-band absorbance ratio shifts at 568 nm and 558 nm wavelengths in individual sample wells measured during oxygen depletion. FIG. 5C is a graph depicting dissolved oxygen measurement over time. Each line in FIGS. 5A-B represents data from an individual well.

FIG. 6 is a graph depicting oxygen concentration corresponding to 50% saturation. Percent saturation was calculated by max-min scaling of the log ratio of absorbance at 568 and 558 nanometers. Because of the limited number of data points due to the one-minute plate scan time (left graph), spline interpolation (right graph) was used to determine oxygen concentration closest to 50% saturation.

FIG. 7 is a flowchart depicting the steps taken to obtain oxygen affinity measurements after a well plate with samples is inserted into a spectrophotometer.

### DETAILED DESCRIPTION

SODA is a process for determining oxygen binding affinity to hemoglobin by measuring light absorbance by RBC samples in liquid suspension under varying atmospheric oxygen levels. The process employs (1) a spectrophotometer that analyzes light passing through liquid RBC samples, (2) at least one probe that measures oxygen levels (and, in some embodiments, also temperature) in the sample liquid, (3) a computer to control the spectrophotometer and at least one probe, and (4) processing to synthesize data exported by recording the measurements from the spectrophotometer and the at least one probe.

A multiwell plate-reading spectrophotometer (PRS) was used to perform rapid light absorbance data capture on multiple RBC samples in a single session. The process was prototyped using a BMG Labtech SpectroSTAR Nano PRS. This device featured a port for introducing compressed gas flow. Compressed nitrogen gas flow displaced room atmosphere oxygen from the PRS. The PRS was placed in an enclosure to control the surrounding atmosphere to stabilize the oxygen concentration. Oxygen displacement causes characteristic light absorbance changes in RBC samples. This PRS featured an internal heating unit which maintains samples near 37 degrees Celsius during experiments to promote normal physiologic oxygen handling by RBCs. Light absorbance readings were taken at regular intervals (e.g., every 60 seconds) throughout the duration of the change in oxygen level from ambient to nearly zero. Data from the PRS was exported for offline analysis.

A 3D printer was used to make custom 24-well plates from polycarbonate filament. The design incorporated conduits for a probe configured to measure oxygen and temperature. A 1 mm polycarbonate sheet was adhered to the bottom of the printed plate body. Stock 24-well plates were modified using custom 3D printed drilling guides. Apertures were made for the probe. Micropipette tips were inserted as guides for the probe.

Precise oxygen levels were recorded in parallel with light absorbance experiments. The SODA process was prototyped using a PreSens OXY1-ST device with a water-compatible fiber optic oxygen probe. The probe was immersed in sample plate wells adjacent to wells containing RBC suspensions. An identical sample buffer was used for RBCs samples, oxygen recording, and buffer temperature recording. The oxygen data was exported for offline analysis.

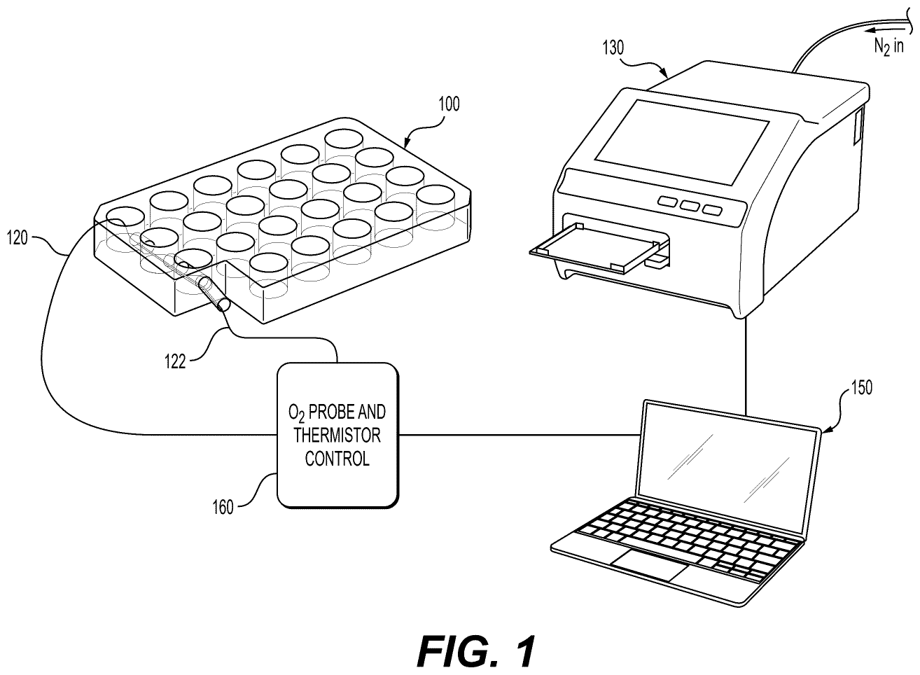

As shown in FIG. 1, an exemplary system may determine oxygen binding affinity for hemoglobin. The system may comprise a plate-reading spectrophotometer (130) configured to analyze light passing through a liquid red blood cell sample. The spectrophotometer (130) may function as a plate reader defining an enclosure, wherein nitrogen flow may be employed to displace atmospheric oxygen. The system may additionally comprise at least one probe (120, 122) in a well plate (100). The at least one probe may be an oxygen probe fiber optic lead configured to measure oxygen level in a liquid sample adjacent to an RBC suspension. In some embodiments, the at least one probe (120, 122) measures oxygen alone. In other embodiments, the at least one probe (120, 122) measures both oxygen and temperature in the liquid sample. The at least one probe may be in communication with a central probe control (160). In certain embodiments, the at least one probe (120, 122) may be disposed in a well plate (100) configured to receive the at least one probe (120, 122). The system may additionally comprise a computer processor (150) that processes data exchanged to and from the spectrophotometer (130) and the probe control (160) and collects data files. Employing SODA processing, the system may be configured to determine oxygen binding affinity for hemoglobin of the red blood cell sample based on the oxygen level and the absorbance data.

Conventional multiwell sample plates do not provide a place to insert the wired oxygen probe that will not interfere with PRS plate handling. Computer-aided design and 3D printing were used to fabricate two solutions: (1) custom multiwell plates with ports for the oxygen probe and (2) custom drilling jigs to create ports for the oxygen probe in standard multiwell plates.

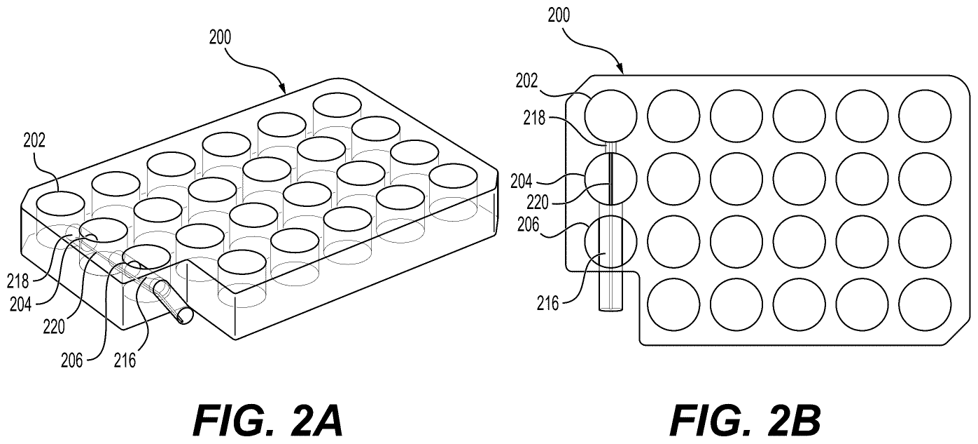

With respect to point (1) above, as shown in FIGS. 2A-B, a stock 24-well plate 3D printing template (200) was modified for use in SODA analysis. As shown in FIGS. 2A-B, a well at Column 1, Row D, was removed to leave space for an oxygen probe (e.g., a single fiber optic filament) (220). The wells at Column 1, Rows A (202), B (204), and C (206) were modified with an angled passage (216) such that an oxygen probe (220) would be supported by the walls of the well at Column 1, Row C (206), and immersed in buffer solution in the well at Column 1, Row B (204). The well at Column 1, Row A (202), was modified with a small, angled channel (218) for the introduction and support of the oxygen probe (220). The oxygen probe (220) feeds through channels (216, 218) into wells (204, 202). The bottomless plates were fabricated using a 3D printer with polycarbonate filament. After fabrication, the printed plate was attached to a transparent base plate using a silicone adhesive. The base plate was cut to the footprint of a standard multiwell plate.

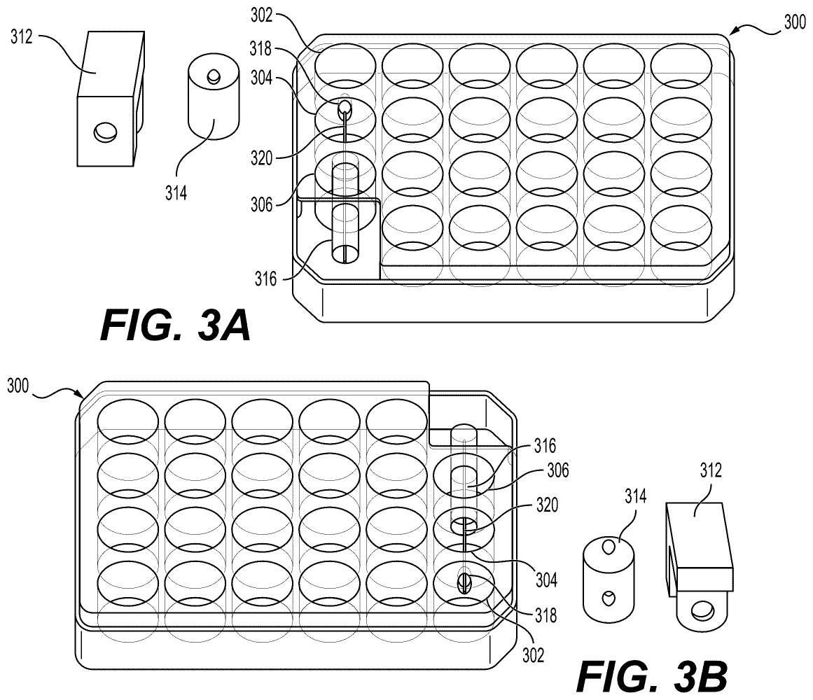

With respect to point (2) above, as shown in FIGS. 3A-B, two drilling jigs (312, 314) were created in CAD software for the purpose of modifying standard 24-well plates. As shown in FIGS. 3A-B, standard plates (300) were first altered by removing a well at Column 1, Row D, using a rotary cutting tool. The first, larger jig (312) was placed in the well at Column 1, Row C (306), to permit drilling an angled passage (316) through the walls of the wells at Column 1, Rows B (304) and C (306). The second, smaller jig (314) was placed in the well at Column 1, Row B (304), to permit drilling a steeply angled passage (318) into the well at Column 1, Row A (302). The passages were fitted with cannulae to support an oxygen probe (320).

SODA processing of the processor (150) (1) synchronizes the PRS and oxygen probe data, (2) correlates changes in light absorbance as oxygen levels change, (3) plots the data correlations, and (4) calculates red blood cell affinity for oxygen. The SODA process was prototyped using specified pieces of hardware with unique capabilities and high-performance standards. The prototype version of SODA software was designed around performance characteristics of the specified equipment. The processing was adapted for blood oxygen affinity analysis with different equipment providing a similar function.

Methods The SODA assay may be formed from an optical plate reader to accommodate oxygen displacement and measurement. Standard 24-well cell culture plates may be modified using a rotary cutting tool and a 3D printed drilling jig to enable connection of an external oxygen probe with temperature sensor (OXY-1 ST, PreSens) to one of the culture wells. The probe provides a high-resolution measure of dissolved oxygen and temperature in a liquid sample. The plate reader and oxygen probe may be controlled by a shared computer to ensure time synchronization.

A custom plate was designed and fabricated to enable simultaneous oxygen measurement during spectra acquisition. The sample plate was loaded into the plate reader, and the plate reader was enclosed in a container to partition the system from surrounding room air. The plate reader was placed in an enclosure, and nitrogen flow was used to displace atmospheric O₂. Light absorbance was measured at regular time intervals for 30 min at ˜37 degrees C. Plates were agitated at 400 RPM in between reads to maintain RBC suspension. After 4-5 baseline plate readings, N₂ gas flow (10 L/min) displaced room air from the plate reader. Light absorbance and dissolved oxygen data were analyzed. A traditional oxygen affinity system was used to validate p50 calculations in cohort RBC samples. Dissolved oxygen was measured in the well plate at different nitrogen flow rates to achieve a controlled oxygen decrease over 30 minutes. Absorbance spectra were acquired with the plate reader while nitrogen flow was introduced. Changes included a shift in the large Soret peak and changes to the Q-band absorbance ratio.

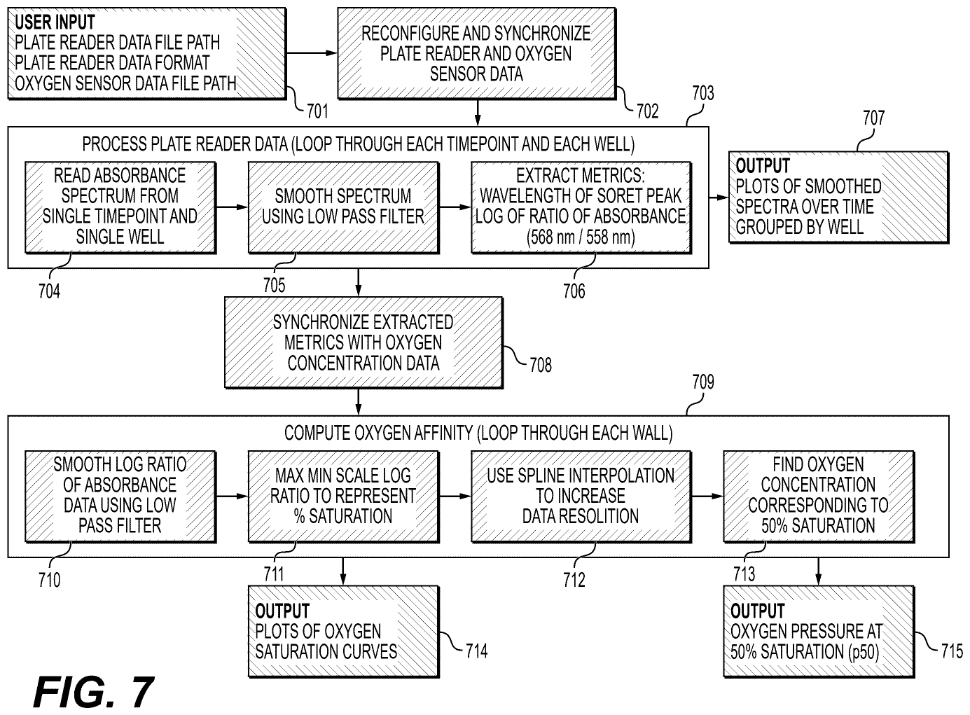

Adult donor RBCs were obtained from commercial sources. Samples were resuspended to 55 hematocrit in AS-3 storage supplement. RBC samples were diluted in PBS and loaded on the modified plate at 1 ml per well. Exact cell concentrations were quantified by hand using a hemocytometer. A TCS Scientific Hemox analyzer was used to calculate traditional oxygen affinity. 30E6 RBCs were used for each Hemox calculation. A PRS was used to perform SODA light absorbance readings. A PreSens sensor system comprising an oxygen probe was used to measure dissolved oxygen and, optionally, also temperature. Data was measured over a period of oxygen depletion, where nitrogen was employed to displace oxygen in an enclosure surrounding a plate reader over a period of about 15 minutes. The PRS generated absorbance spectra as a function of wavelength for each well of the plate at each timepoint. PRS and oxygen probe data were exported to csv files and analyzed. FIG. 7 depicts a flowchart showing the steps taken to analyze each plate and generate graphical and numerical data. Briefly, the user inputs the file locations for the plate reader and oxygen probe data and the system generates data plots and computes oxygen affinity as oxygen pressure at 50% hemoglobin saturation, or p50. In particular, user input may comprise a path or filename for plate reader data, a plate reader data format, a path or filename for oxygen probe data, an oxygen probe data format, and wells in a well plate that contain a liquid for analysis (701). The analysis may comprise importing and reconfiguring plate reader data and, separately, oxygen probe data (702). Processing of plate reader data (703) may comprise, for each timepoint and each well, reading an absorbance spectrum (704), smoothing the spectrum using a low pass filter (705), detecting a Soret peak and recording its wavelength (706), measuring absorbance value at wavelengths of interest (e.g., 558 nm and 568 nm), and computing the log of the ratio of absorbance at 568 nm to 558 nm (706). Processing of plate reader spectrum data may further comprise displaying smoothed spectra grouped by well (e.g., each plot contains spectra from all timepoints for that well) (707) and determining oxygen concentration for each timepoint by matching time of each spectrum with oxygen probe data (708). Computing and displaying metrics for each well may comprise displaying Soret peak wavelength and log ratio of absorbance vs. timepoint and/or vs. oxygen concentration. Computing oxygen affinity (as p50 value) (709) may comprise smoothing the log ratio of absorbance data using a low pass filter (710), using max-min scaling to transform the absorbance ratio data to a 0-100% scale (711), thus generating a oxygen saturation curve (714), using spline interpolation to increase data resolution near the 50% saturation value (712), and determining the oxygen concentration at 50% saturation (713) to obtain a p50 value (715).

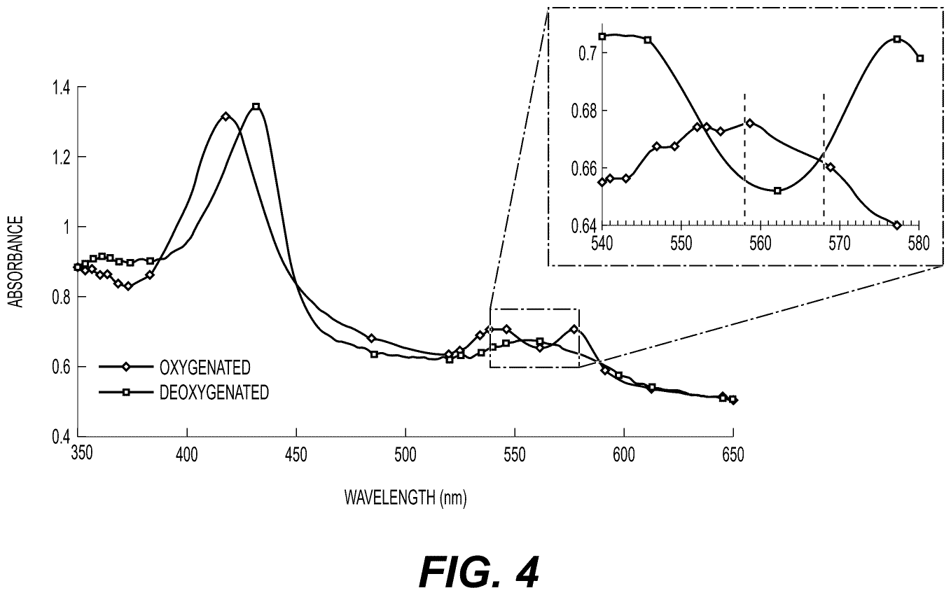

Absorbance spectra were acquired with the plate reader while nitrogen flow was introduced. Changes included a shift in the Soret peak, as well as changes to the Q-band absorbance ratio. FIG. 4 shows an example of acquired spectra in fully oxygenated and deoxygenated states. As can be seen, the depicted large Soret peak shifted to the right. The enlarged inset depicts changes in the Q-band that were leveraged for oxygen affinity calculations.

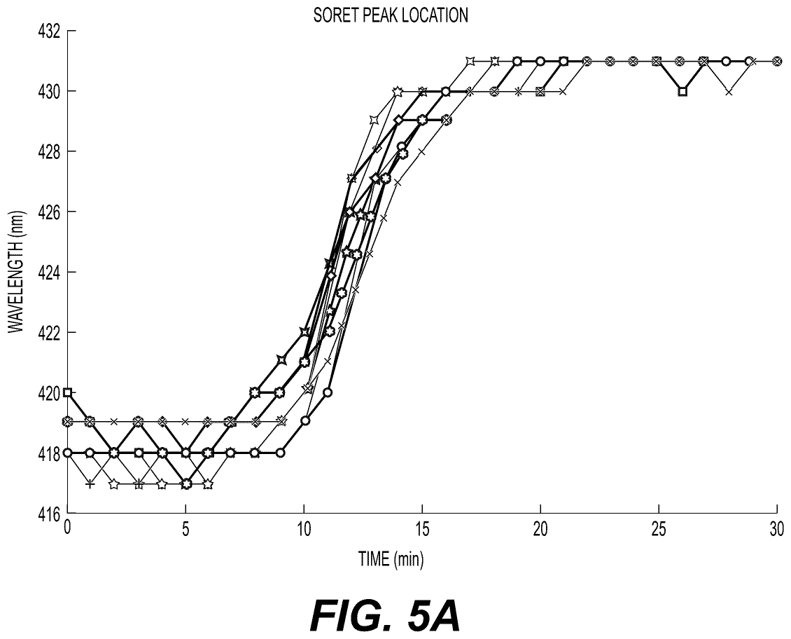

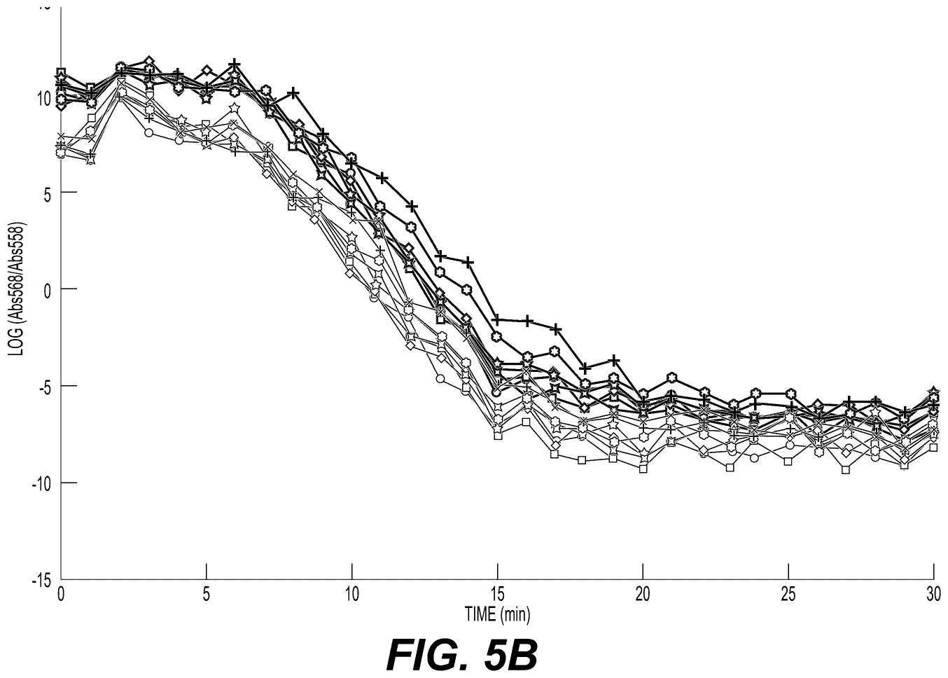

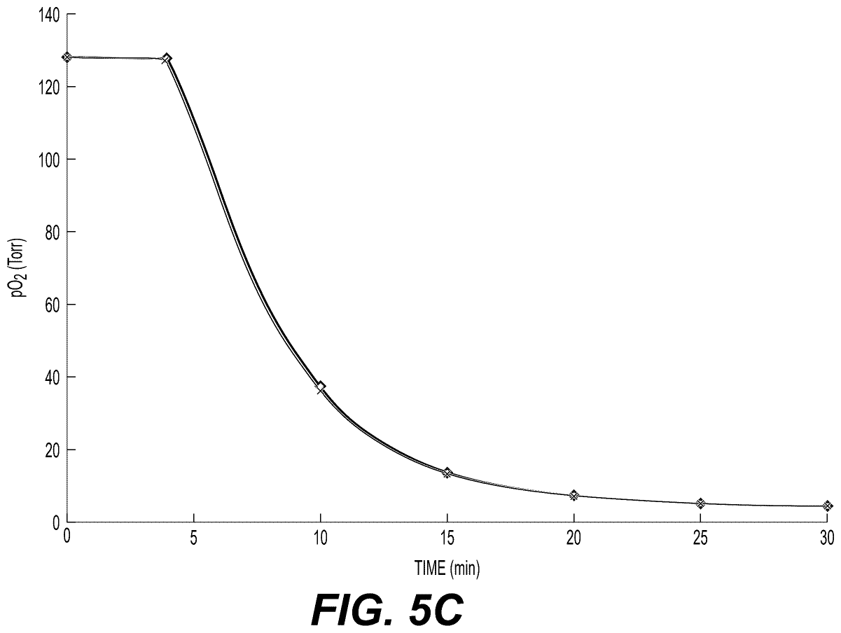

The SODA analysis performed by the processor (150) reads in the PRS and oxygen probe data. The PRS data contains a full spectrum of absorbance data, typically ranging from 350 nm-700nm, for every timepoint and every well of the plate. The SODA processing automatically looped through all timepoints and wells to extract key features from the spectra. The SODA processing smoothed each spectrum using a low-pass filter. SODA determined the wavelength of the Soret peak maxima (415-430 nm). SODA then calculated the log of the ratio of absorbance at the Q-band isobestic point (568 nm) to absorbance at the Q-band variable point (558 nm), the two wavelengths employed in standard Hemox Analyzer measurement. The system may provide automated processing of spectra across wells in a well plate. Spectra were acquired every minute with nitrogen flow initiated after a settling period. Soret band shifts (FIG. 5A) and Q-band absorbance ratio shifts (FIG. 5B) were measured during dissolved oxygen depletion (FIG. 5C).

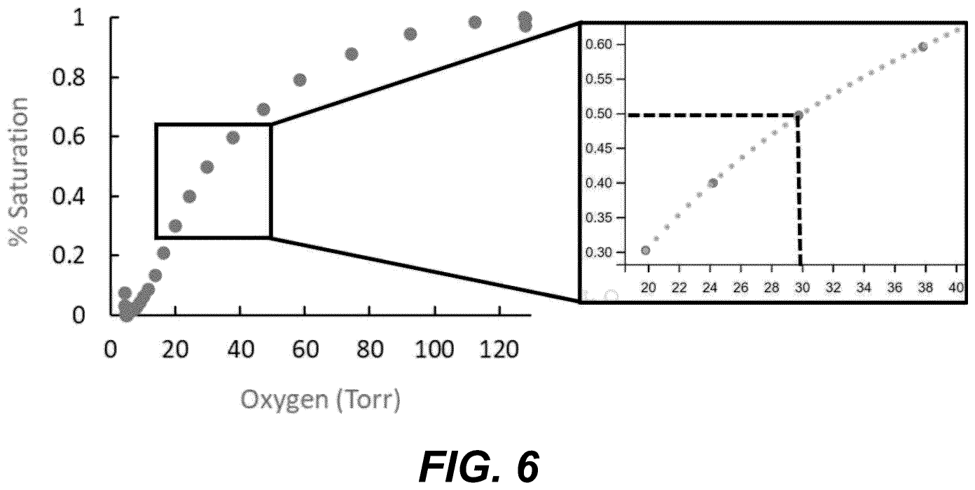

For each well, the log ratio of absorbance at 568 nm to 558 nm was calculated as a function of time (FIG. 5B). The ratio data was smoothed using a low-pass filter followed by a max-min scaling to represent percent saturation. The scaled ratio was plotted as a function of oxygen concentration by aligning the time on the oxygen probe data. To determine a value analogous to p50 on a Hemox Analyzer, spline interpolation was employed to increase the resolution of data. The oxygen concentration using torr as a measuring unit corresponding to a 50% saturation point was determined, as reflected in the graph shown in FIG. 6. In FIG. 6, p50 was calculated from the Q-band absorbance ratio. Percent saturation was calculated by max-min scaling of the log ratio of absorbance. Because of the limited number of data points due to a one-minute plate scan time (left graph), spline interpolation (right graph) was employed to determine oxygen concentration closest to 50% saturation.

### Results

Systems and methods in accordance with embodiments of the present disclosure provide automated processing of the spectra across the wells. Spectra were acquired every minute with the nitrogen flow initiated after a settling period. p50 was calculated from the Q-band absorbance ratio. For comparison, donor blood samples were analyzed with the SODA assay and the traditional Hemox analyzer. p50s calculated from SODA similarly showed the pH effect and the aging effect. Absolute values were somewhat dependent on cell density, suggesting that additional correction factors are needed for quantitative matching.

Adult donor RBCs produced classical, stereotyped light absorbance patterns in the visible spectra between 400 and 600 nm λ. Increasing cell concentration caused nonlinear increases in absorbance (A) across the spectrum, but especially in 350 to 500 nm. Oxygenated RBCs exhibited the Q-band doublet with maxima at 541 and 577 nm, while deoxygenated RBCs exhibited a single maximum at 560 nm.

### Conclusion

SODA was developed on an equipment platform that is relatively low cost and finds general laboratory use beyond RBC experiments. RBC Hb can be desaturated and resaturated with oxygen in a controllable and quantifiable manner in this system. SODA results are reliable across a range of RBC dilutions and can account for variability in sample loading. Basic principles of Hb function, such as pH sensitivity and allosteric modulation, can be analyzed using this system. SODA could prove valuable to blood banks seeking capability to rapidly census their supplies for potency during storage aging. Artificial blood substitute development could benefit from SODA analysis if these products exhibit light absorbance changes during desaturation.

Conventional, state-of-the-art technology does not include the original back-end, experimental analysis of SODA. SODA can access plate reader data files and make customized calculations to analyze data. Indeed, a mature form could be a benefit to other groups'processes.

SODA is distinct from state-of-the-art technology, such as Patel et al. (2018) (“Patel”) and Woyke et al. (2021) (“Woyke”). Patel breaks open red blood cells to release hemoglobin into a buffer solution. Intact cells scatter light, potentially confounding biochemical analysis. Patel employs simple arithmetic algorithms in Microsoft Excel for analysis of spectrophotometer results. Free hemoglobin measures differently relative to hemoglobin in intact red blood cells. SODA assesses intact blood cells to preserve biologic fidelity of hemoglobin function.

Woyke designed a novel 96-well plate that facilitates tight control of oxygen levels in liquids supporting blood cells, while miniature wireless oxygen sensors in the wells relay real-time oxygen concentrations. Woyke examines small quantities of red blood cells, and measures cell sample green light absorbance. Similar to Patel, Woyke employs simple arithmetic algorithms in Microsoft Excel for analysis. SODA uses standard 24-well plates with minor modifications to allow the use of a wired oxygen probe. SODA collects all wavelengths of light from ultraviolet to infrared and analyzes red light absorbance data similar to the industry standard Hemox Analyzer. Green light absorbance, such as that measured in Woyke, is drastically altered by sample cell concentration and thus less reliable when assessing oxygen affinity.

In summary, SODA is a variation on a process for analyzing red blood cell oxygen binding. SODA's unique contribution is the creation of a new, novel system and method for analyzing native blood cell light absorbance data.

What is claimed is:

1. A method of determining oxygen binding affinity to hemoglobin, comprising measuring light absorbance by red blood cell (RBC) samples in a liquid suspension.

2. The method of claim 1, further comprising:
analyzing, via a plate-reading spectrophotometer (PRS), light passing through a liquid RBC sample;
sensing, via at least one probe, oxygen level of the RBC sample, the at least one probe being disposed in a well plate configured to receive the at least one probe; and
determining oxygen binding affinity for hemoglobin of the RBC sample based on the oxygen level and absorbance data measured by the PRS.

3. A system for determining oxygen binding affinity for hemoglobin, comprising:
a plate-reading spectrophotometer (PRS) configured to analyze light passing through a liquid RBC sample;
at least one probe, the at least one probe configured to measure oxygen level of the RBC sample and being disposed in a well plate configured to receive the at least one probe; and
a processor configured to determine oxygen binding affinity for hemoglobin of the RBC sample based on the oxygen level and absorbance data measured by the PRS.

4. The method of claim 2, wherein the well plate includes wells arranged in columns and rows, and wherein one of the columns and one of the rows has a number of wells smaller than a number of wells in other columns and rows, respectively.

5. The method of claim 2, wherein at least two wells of the well plate define a respective passage that receives the at least one probe.

6. The method of claim 2, wherein at least one well of the well plate defines an angled port that receives the at least one probe.

7. The method of claim 2, wherein the well plate is fabricated from a 3D printed polycarbonate filament.

8. The method of claim 2, wherein the well plate is attached to a transparent base plate using a silicone adhesive.

9. The method of claim 2, further comprising sensing, via the at least one probe, temperature of the RBC sample.

10. The method of claim 2, wherein the absorbance data is read from a single timepoint in a single well in the well plate and a smooth spectrum of the data generated using a low pass filter.

11. The method of claim 2, wherein the oxygen binding affinity is determined by smoothing a log ratio of the absorbance data using a low pass filter, employing a max-min scale log ratio to represent a percent saturation of oxygen and spline interpolation to increase data resolution, and determining an oxygen concentration that corresponds to 50% oxygen saturation.

12. The system of claim 3, wherein the well plate includes wells arranged in columns and rows, and wherein one of the columns and one of the rows has a number of wells smaller than a number of wells in other columns and rows, respectively.

13. The system of claim 3, wherein at least two wells of the well plate define a respective passage that receives the at least one probe.

14. The system of claim 3, wherein at least one well of the well plate defines an angled port that receives the at least one probe.

15. The system of claim 3, wherein the well plate is fabricated from a 3D printed polycarbonate filament.

16. The system of claim 3, wherein the well plate is attached to a transparent base plate using a silicone adhesive.

17. The system of claim 3, wherein the at least one probe is configured to measure temperature of the RBC sample.

18. The system of claim 3, wherein the absorbance data is read from a single timepoint in a single well in the well plate and a smooth spectrum of the data generated using a low pass filter.

19. The system of claim 3, wherein the oxygen binding affinity is determined by smoothing a log ratio of the absorbance data using a low pass filter, employing a max-min scale log ratio to represent a percent saturation of oxygen and spline interpolation to increase data resolution, and determining an oxygen concentration that corresponds to 50% oxygen saturation.
